(19) United States
(12) Patent Application Publication
(10) Pub. No.: US 2020/0154790 A1
(43) Pub. Date: May 21, 2020

(54) Children's Training Pants and Method of Use

(71) Applicant: Maria Helena Cleary, Denver, CO (US)

(72) Inventor: Maria Helena Cleary, Denver, CO (US)

(21) Appl. No.: 16/686,047

(22) Filed: Nov. 15, 2019

Related U.S. Application Data
(60) Provisional application No. 62/768,553, filed on Nov. 16, 2018.

(51) Int. Cl. A41B 9/00; A61F 13/496

(52) U.S. Cl. CPC A41B 9/001; A61F 13/66; A61F 13/496

(57) ABSTRACT

According to the invention, a children's training pant for preserving diaperless feel during bare a bottom training session is composed of an outer modesty pant sized to fit a child and formed of a light weight, quick dry fabric. An inner liner pant is sized to fit a child, is formed of net fabric, and is configured when worn to overlie a user child's bottom region and crotch area. A removable absorbent pad is adhered within said inner liner pant over a crotch covering area thereof.

### BACKGROUND OF THE INVENTION

### Field of the Invention

The invention generally relates to means and methods for collecting body fluids, and particularly to a pants-like support for an absorbent pad. The support has a specific design, shape, or structural feature. The invention also relates to absorbent articles specifically adapted to be worn around the waist, and particularly to reusable articles of this type. The invention is directed to an absorbent garment, such as a training pant, with adaptation of use in original toilet training of children.

### Description of Related Art Including Information Disclosed Under 37 CFR 1.97 and 1.98

Toilet training of children is a unique process because it is original training for which the child has no background. Thus, this training is distinctly different from handling adults with incontinence. The child is reasonably expected to learn and master bodily control functions, where the adult has lost this ability due to age, physical degeneration, or the like, and is not reasonably expected to again master the lost bodily control functions. With adults, the solution is to use absorbent diaper-like pads that simply contain excrement. However, children are different because from birth they have no recognition or control of toilet functions. When undergoing original training, the solution is in acclimating the child to recognize impending excretory functions then following through with toilet usage.

There are numerous methods for toilet training children. The present method Is fast and effective because it employs a solution to a related and often unrecognized inhibition in the child's immediately prior life and the obscuring of sensation. It would be desirable to provide a toilet training aid that comports with the child's prior training experience and continues inhibition fueled toilet training.

To achieve the foregoing and other object and in accordance with the purpose of the present invention, as embodied and broadly described herein, the method and apparatus of this invention may comprise the following.

### BRIEF SUMMARY OF THE INVENTION

Against the described background, it is therefore a general object of the invention to improve upon a currently favored method of allowing a child in training to go “bare bottom” and monitor the child 24/7. This method allows the child to experience and acclimate to nakedness. The child loses the experiences of protection and acclimates to the sensation of having no protection as previously supplied by the presence of a diaper or the like. So long as the sensation of nakedness is maintained, the way is clear for the child to feel inhibition against uncontrolled toilet habits and to learn control.

The present invention builds upon the “bare bottom” approach. Since the “bare bottom” approach requires constant monitoring, the practicality of the approach is limited by parent's or caretaker's schedule and need to attend to other aspects of life, such as going back to work. Thus, after attempting the “bare bottom” approach for a few days, such as a weekend, the parents often regress to using diapers on the child. With this regression, the child reverts to a familiar protected mode that ignores the “bare bottom” training. The inhibition against uncontrolled toilet habits can be lost and may need to be repeated in the future.

The invention is directed to a pant-like garment with absorbent capacity. The novelty of the garment is in a construction that is non-pressing on the child's body, so that despite wearing the garment, the child largely continues to sense naked or “bare bottom” status. Thus, the child's training according to the “bare bottom” method continues despite the interruption in actual “bare bottom” condition. The pant-like garment still mitigates the most serious effects of accidents.

With the foregoing in mind, it is a feature and advantage of the invention to provide a pant-like garment that is made of a combination of features, which may be selected from extremely light materials and construction to be substantially non-binding and non-compressed against the child's body, such that the garmet is substantially non-detectable or minimally detectable by the child so that it preserves the child's sensation of being unprotected over the area covered by the pants.

According to the invention, a children's training pant for preserving diaperless feel during bare a bottom training session is composed of an outer modesty pant sized to fit a child and formed of a light weight, quick dry fabric. An inner liner pant is sized to fit a child, is formed of net fabric, and is configured when worn to overlie a user child's bottom region and crotch area. A removable absorbent pad is adhered within said inner liner pant over a crotch covering area thereof.

The accompanying drawings, which are incorporated in and form a part of the specification, illustrate preferred embodiments of the present invention, and together with the description, serve to explain the principles of the invention. In the drawings:

### BRIEF DESCRIPTION OF THE SEVERAL VIEWS OF THE DRAWINGS

FIG. 1 is a front elevational view of an outer loose fitting modesty garment, showing both elastic waist and leg or alternative cord secured waist or leg.

FIG. 2 is a front elevational view of a large mesh net inner garment.

FIG. 3 is a front elevational view of an adhesive-mounted absorbent pad fitting the net garment of FIG. 2.

FIG. 4 is a front elevational view of the components of FIGS. 1-3 assembled with transparency of nearer components to show underlying components, and showing alternative tape secured waist or leg.

FIG. 5 is detail of an leg opening showing an example of a substantially pressure free rib as an alternative to an elastic loop at a leg or waist opening.

### DETAILED DESCRIPTION OF THE INVENTION

The invention is a multi-component training pants to be worn like underpants by a child undergoing “bare bottom” toilet training. According to this popular method of training, a child Is allowed to spend his days with “bare bottom” until the child learns to control his toilet functions. The reasoning behind this method is that from birth a child first becomes accustomed to the feeling of a diaper and is comfortable to use the diaper without conscious concern or control about what might be called “potty events.” The diaper provides a level of protection that has been present since the child's birth. As long as this protection is present, the child has no concerns about potty events and may be slow to overcome habit.

When toilet training begins, the child is signaled of a change by the absence of the protection afforded by the diaper. Without the diaper, the child will notice the absence of the familiar sensation of wearing the diaper, and any potty events will be felt differently. Parents or caretakers, who will be referred to as adults, need to continuously monitor the child during bare bottom training so that any potty events are promptly cleaned up.

A problem with bare bottom training is that few adults enjoy extended numbers of days to monitor the child's progress. A time may come when adult's piece of mind may compel him to re-diaper the child, temporarily at least. The return of the diaper may inspire the child to regress in his toilet training and again to enjoy the comfort of uncontrolled potty events. This is an undesirable step back to habit and is solved by the present invention.

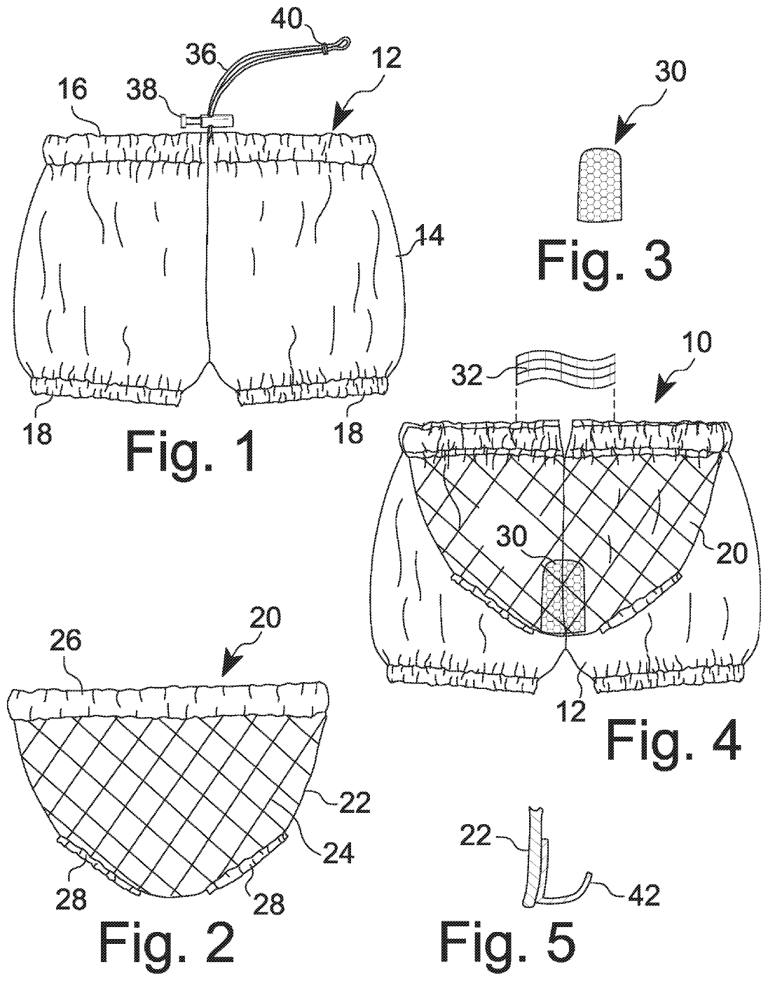

With reference to FIGS. 1-4 of the drawings, the invention is a training pant garment 10 that provides almost no sensation to the child, while still providing a small degree of protection against accidental potty events. Should the adults need to equip the child with protection, the garment 10 meets this need while minimizing the likelihood that the child will regress. Several factors are at work to prevent regression. First, the garment 10 minimizes physical contact with the child's body. Second, the garment 10 is of exceedingly light weight so as not suggest the feeling of a diaper. Third, the garment 10 is designed to retain only a small quantity of excrements, so that the child does not experience the same feeling as when using a diaper.

In FIG. 1, an outer modesty pant 12, referred to herein as a “bloomer,” is shown to be loose fitting and considerably wider than the child's midsection or hip so that the outer bloomer garment does not compress inner garments against the child's body. As a sizing guide but not a limitation, the center of pant body 14, at the hip of a suitable bloomer 12, is four or more inches in circumference greater than the child's hip demension. Conveniently, using standard garment sizing for children, the bloomer should be sized at least four inches greater than any specified hip dimension for a child's pant of a specified size. This outer garment 12 is similar to known “bloomers” but with baggy design. The component fabric is light weight. Ripstop nylon is a suitable light weight fabric. Other choices include polyolefin, polyester, polyamide, or mixtures thereof. It is desirable that the chosen fabric is washable and quickly dries so that recovery from any potty event is quick. The bloomers 12 can be assigned a children's clothing size corresponding to the child's normal age and physical characteristics. With consideration of such sizing, the bloomers 12 might be given an elastic loop waist 16, as well as elastic loop leg openings 18. The chosen elastic should be light and low tension so as to chosen and sized to minimize pressure sensation to the child.

In FIG. 2, an inner garment 20, referred to as a “liner,” serves as a carrier for an absorbent pad. This liner is a pant worn next to the child's body and is uniquely configured to avoid feeling like a diaper. The body 22 of the liner is not required to duplicate the modesty function of the bloomer 12. Hence, the body 22 is formed of a net or mesh material formed of supporting strands 24. Such a net of mesh fabric is both light weight and extremely open to air circulation and will contribute to a non-diaper feeling for the child. The inner pant 20 may be configured as a pant-like garment, or it may be reduced to sufficient holders to support a pad. Where the liner pant is used, the waist 26 and leg openings 28 can be sized by light weight, low tension elastic. As with the bloomer 12, the elastic should be chosen and sized to minimize pressure sensation to the child.

With reference to FIG. 3, the absorbent pad 30 can be equipped with an adhesive side for placement in the crotch of the liner 20. Pads of this type are commercially available. In this application, the pad provides minimal protection against accidental potty events.

In minimizing diaper feel with the bloomers 12 and liners 20, alternatives can be used in lieu of the elastic loops at waists 16 and 26 and leg openings 18 and 28. One suitable substitute suggested in FIG. 4 is tape 32 used to hold together open seam edges 34 at the waist or leg without applying pressure. This solution can provide a precision, nonbinding waist or leg opening and add lightness to the engagement of the garments with the child. Another alternative solution is an elastic or inelastic draw string 36 shown in FIG. 1, which can be precisely adjusted by tying a knot 40 or by sliding an adjustable cord clip 38 to closely fit the child at the leg or waist without applying pressure. Still another solution is to edge the garment body 22 at waist and leg openings with a flexible rib 42 shown in FIG. 5. Any of these alternatives to an elastic loop can be essentially pressure free, preserving the impression of being diaper-free.

When child no longer sense the early security of a diaper, he will gain awareness of toilet functions. With this new awareness, the child will gravitate toward the new training solution. The invention will provide a continuation of bare bottom training methods through times when a bare bottom status is not available.

The foregoing is considered as illustrative only of the principles of the invention. Further, since numerous modifications and changes will readily occur to those skilled in the art, it is not desired to limit the invention to the exact construction and operation shown and described, and accordingly all suitable modifications and equivalents may be regarded as falling within the scope of the invention as defined by the claims that follow.

What is claimed is:

1. A children's training pant for preserving diaperless feel during a bare bottom technique training session, comprising:
an outer modesty pant sized to fit a child and formed of a light weight, quick dry fabric;
an inner liner pant sized to fit a child, formed of net fabric, and configured when worn to overlie a user child's bottom region and crotch area; and
a removable absorbent pad adhered within said inner liner pant over a crotch covering area thereof.

2. The children's training pants of claim 1, wherein:
said inner liner pant has waist and leg openings with a sizing adjuster for fitting a child with preservation of diaperless feel, the sizing adjuster being selected from the group consisting of a draw string, a draw string with position adjustable clip, tape, and an edge rib.

3. The children's training pant of claim 1, wherein:
said modesty pant is sized with a child's garment size having a known hip specification; and
the modesty pant is configured to be at least four inches in circumference greater than the hip specification for said child's size.

4. A children's training pant for preserving diaperless feel during a bare bottom technique training session, comprising:
a pant sized to fit a child, formed of net fabric, and configured when worn to have a crotch covering area positioned in an anticipated location to overlie a user child's crotch area; and
a reception area at an inner location of said pant, at said crotch covering area thereof.

5. The children's training pant of claim 4, wherein:
said pant has waist and leg openings with a sizing adjuster for precisely fitting a child with preservation of diaperless feel, the sizing adjuster being selected from the group consisting of a draw string, a draw string with position adjustable clip, tape, and an edge rib.
